(19) United States
(12) Patent Application Publication
(10) Pub. No.: US 2005/0048571 A1
(43) Pub. Date: Mar. 3, 2005

(54) Porous glass substrates with reduced auto-fluorescence

(76) Inventors: Paul S. Danielson, Corning, NY (US); Jodelle K. Montondo, Painted Post, NY (US)

Correspondence Address: CORNING INCORPORATED, SP-TI-3-1, CORNING, NY, 14831, US

(21) Appl. No.: 10/629,444

(22) Filed: Jul. 29, 2003

(51) Int. Cl. G01N 33/53; C12M 1/34

(52) U.S. Cl. 435/7.1; 435/287.2

(57) ABSTRACT

A porous inorganic substrate that may be used as a support, upon which biological, biochemical, or chemical reactions may be executed, is provided. A porous layer of the substrate is tinted with a colorant agent, and the porous substrate has at least about 15-20% less auto-fluorescence background relative to a conventional “white” porous substrate. The tinted porous layer provides improved signal to noise ratios that are important in detection metrics when performing biological or chemical binding assays. Once the porous layer is functionalized, one may immobilize probe molecules on or within the porous layer to create a microarray having a greater probe concentration and retention capability than conventional non-porous inorganic substrates, without suffering relatively high levels of auto-fluorescence and other detriments common to un-tinted porous substrates.

### FIELD OF INVENTION

The present invention relates to a glass material that forms at least a part of a porous substrate used for biological or biochemical assays. In particular, the invention pertains to material compositions that exhibit reduced levels of auto-fluorescence under certain light wavelengths.

### BACKGROUND OF THE INVENTION

In recent years, solid-phase microarray technology has blossomed. Biological, pharmaceutical, and other research communities have recognized that microarrays are useful, high-throughput research tools to measure a variety of biological or biochemical functions. With widespread acceptance, the microarray format is likely to remain a key research tool into the foreseeable future. Applications for microarray technology will continue to expand in the areas of drug discovery and development, diagnostic assays, and biological research.

Biological or chemical probe molecules can be immobilized on a solid surface for many kinds of assays. For instance, high-density arrays have become invaluable tools for drug researchers and geneticists in a variety of binding assays, such as to obtain information on the expression of genes. One may monitor changes in gene expression in single nucleic acid polymorphism (SNP) assays using a microarray containing nucleic acid analytes. Clinical and research laboratories are increasingly using DNA testing as a means to determine genetic risk factors for diseases like breast cancer, heart disease, Alzheimer's disease, etc. Simultaneous screening for many risk factors is possible by printing many “microdots” of DNA onto the same substrate, typically either a porous, organic membrane or a flat, non-porous glass slide to form a high-density array. A high-density array typically comprises between 2,000 and 50,000 probes, with the possibility of up to about 80,000 or 100,000 probes, in the form of single stranded DNA, each of a known and different sequence, arranged in a predetermined pattern on a substrate.

After exposing the array to target molecules, such as nucleic sequences under selected test conditions, scanning devices can examine each location on the array and determine the quantity of targets that are bond to complementary probes. The ratio of fluorescent intensity relative to a reference at each spot on the high-density array provides the relative differential expression for a particular gene. DNA arrays can be used to study the regulatory activity of genes, wherein certain genes are turned on or “up-regulated” and other genes are turned off or “down-regulated.” So, for example, a researcher can compare a normal colon cell with a malignant colon cell and thereby determine which genes are being expressed or not expressed in the aberrant cell. The regulatory sites of genes serves as key targets for drug therapy.

Proper performance of a DNA array depends on two basic factors: 1) retention of the immobilized probe nucleic sequences on the substrate, and 2) hybridization of the target sequence to the immobilized probe sequence, as measured by fluorescence emission from the tagged target sequence. The DNA probe material must be retained on the surface of the substrate through a series of washing, blocking, hybridizing, and rinsing operations that are commonplace in DNA hybridization assays. An excessive loss of probe DNA sequences can lead to a low fluorescent-signal-to noise ratio and uncertain or erroneous results.

DNA arrays have for years been printed onto organic, micro-porous membranes such as nylon or nitrocellulose. The densities at which one can print DNA solutions onto these types of organic micro-porous membranes is limited because of the tendency for the DNA solution to wick laterally through the membrane, thus causing cross-talk and contamination between adjacent locations. Others have employed a flat, non-porous substrate surface made from glass. (See for example, U.S. Pat. No. 5,744,305, incorporated herein by reference.) Non-porous, planar, solid substrates, such as microscope slides, coated with a functionalized layer, have been a preferred surface upon which to deposit or print a variety of probe molecules for microarrays.

The organic or non-porous inorganic types of substrates, however, have been found wanting, since they do not retain the probe molecules as well as porous substrates. Recently, researchers have turned to coated, porous substrates to improve detection capabilities under certain light wavelengths or recognition parameters used to analyze the microarrays. Due to an increased total surface area, a three-dimensional, porous substrate can generate greater signal intensity, by about 10 to 25 times more, than a conventional flat two-dimensional, coated slide. A typical porous substrate comprises a base substrate that is either micro-porous or non-porous, and having either pores formed therein, or a porous layer bonded to a top surface of the base substrate. The base substrate is preferably made from a suitable glass.

Although porous glass substrates can generate high signal intensity, they unfortunately also have an increased level of background auto-fluorescence or noise, which can be detrimental to the overall signal to noise ratio. Often during the detection step of an assay, background fluorescence from the substrate's surface under certain light wavelengths can obscure or optically “wash out” the signal emitted from fluorescently labeled binding partners of immobilized probe molecules. A high level of auto-fluorescence in the substrate prevents the user from accurately determining a base-line level of fluorescence. Hence, assay detection and analysis may suffer.

Previous attempts to reduce auto-fluorescence by means other than the addition of colorants were unsuccessful. One example involved darkening the color of porous glass substrates by firing the glass slide in a neutral atmosphere containing no oxygen at about 705° C. The hoped for effect was that the pyrolysis and incomplete oxidation of organic binders during firing would blacken the slide. Rather than darkening, however, an increase in the auto-fluorescence was observed. In a second approach, two previously fired porous glass substrates were fired a second time in a hydrogen-nitrogen atmosphere for 60 hours at about 720° C. Such heat treatments have been used to darken commercial glasses containing reducible ions (e.g., As, Sb, Pb, etc.) The resultant glass's color changed from an opaque white to a translucent brown. The auto-fluorescence background signal was reduced but the porous structure, unfortunately, also was affected. The porous layer glass had fused, losing porosity, hence the H₂-N₂-fired slide had little structural difference from a conventional, two-dimensional flat slide surface.

### SUMMARY OF THE INVENTION

The present invention addresses the issue of high auto-fluorescence or reflectivity in porous substrates without suffering the adverse effects of previous techniques. According to the present invention, a glass frit composition was modified to reduce reflectance in an attempt to decrease the level of intrinsic auto-fluorescence of microarray substrates. Colorant ions were incorporated into the glass composition in order to absorb and reduce stray reflected light, but without seriously reducing the overall desirable fluorescence signal. Using either cobalt or nickel (II) oxides, separately or in a combination with each other or other transition metal species added to a porous glass composition, one can create a tint that reduces reflectance and background signal due to scattering. Relative to “white” or un-tinted porous slides, the net signal for Cy5 and Cy3 labels are both respectively lower with a tinted porous substrates by at least twenty or twenty-five percent. Signal to noise ratio for a tinted porous substrate is dramatically improved over white porous glass or inorganic materials. The reduction in Cy5 generated background can significantly impact the signal to noise metric for microarray analysis.

The present invention, in one aspect, includes a porous substrate having: a support; and a porous region on a surface of said support. The porous region is composed of a primarily inorganic material and having a surface upon which a number of probe molecules can be immobilized. The porous region also has a tint and exhibits a reduction in relative reflectance and auto-fluorescence levels by at least about 15% or 20%, preferably about 50%, over a non-tinted porous substrate surface, over a wavelength range from about 400 or 420 nm to about 700 or 720 nm. The tinted porous region has a colorant component, including a transition metal ion, incorporated into its composition. The tinted porous region may have a composition in weight percent consisting essentially of: 53-67% SiO₂; 3-10% Al₂O₃; 12-24% B₂O₃; 0-5% K₂O; 0-2% MgO; 0.5-3% CaO; 0-3% SrO; 2-7% BaO; 0-2% Sb₂O₃, and at least one of the following, either individually or in combination, 0.1-9% Co₃O₄; 0.1-10% NiO; or 0-10% RₓO_{y} wherein R is a transition metal, and x and y are each ≧0. The R transition metals may include Fe, V, or Cu. More preferably, the porous layer has a composition that consists essentially of: 55-65% SiO₂; 4-9% Al₂O₃; 14-21% B₂O₃; 1-5% K₂O; 0.1-2% MgO; 1-2.5% CaO; 0.5-1.75% SrO; 3-5% BaO; 0-2% Sb₂O₃, and at least one of the following, either individually or in combination, 0.1-8% Co₃O₄; 0.1-10% NiO; 0-10% RₓO_{y}, preferably with the cobalt and nickel species both present. The glass compositions are chemically and mechanically durable, and have a coefficient of thermal expansion (CTE) of between about 35-44×10⁻⁷/° C., preferably 38-40×10⁻⁷/° C., which is suitable to bind securely with typical non-porous glass slides or substrates.

For biological assay applications, the porous substrate is prepared with a number of biological or chemical probes. The probe species or molecules are attached at defined locations on or within the tinted porous layer. A set of probes at defined locations form a microarray of probe microspots having a density of at least one microspot per cm², preferably at least 10 microspots per cm², more preferably about 20-100 microspots or more per cm².

Additional features and advantageous of the present invention will be revealed in the following detailed description. Both the foregoing summary and the following detailed description and examples are merely representative of the invention, and are intended to provide an overview for understanding the invention as claimed.

### BRIEF DESCRIPTION OF THE FIGURES

FIG. 1 is a graphical representation comparing the relative average background fluorescence of an uncoated a tinted porous glass layer on a substrate (I) according to the present invention with a control (A), an un-tinted “white” porous layer (B), and a flat non-porous substrate surface (C), each of which are also uncoated. Table 1 summarizes the data of this graph.

FIG. 2 is a graphical representation comparing the relative mean local background auto-fluorescence of a tinted porous glass layer on a substrate with a control (A), an un-tinted “white” porous layer (B), and a flat non-porous substrate (C), each of which is coated with a layer of 5% gamma-aminopropylsilane (GAPS) and treated with a reducing agent, such as NaBH4. Table 2 summarizes the data of this graph.

FIGS. 3A and 3B are graphical representations of the net fluorescence signals (i.e., mean signal less mean background) for cyanine dyes, Cy5 and Cy3, respectively.

FIGS. 4A, 4B, and 4C are scanned false-color images of three substrates. Serving as a control for comparison purposes, FIG. 4A is an image of a flat plane of a non-porous glass, such as used in flat-panel display or LCD devices. FIG. 4B is an image of a tinted porous glass surface, according to the present invention. FIG. 4C is an image of a prior “white” porous glass surface.

FIGS. 5A and 5B are false color images of arrays that have undergone hybridization, and show, in Cy5-channel wavelength, a comparison between tinted and un-tinted porous layers, respectively, on slides that were not treated with a pre-hybridization buffer containing NaBH₄.

FIGS. 6A and 6B are false color images of arrays that have undergone hybridization, and show, in Cy3-channel wavelength, a comparison between tinted and un-tinted porous layers, respectively, on slides that were treated with a pre-hybridization buffer containing NaBH4.

FIG. 7 is a graph comparing the signal to noise metric for samples of the different types of substrates. As one may observe, the tinted substrate exhibited the highest relative Cy5 signal of any of the substrate types. The signal-to-noise ratio for the tinted porous substrate, untreated with NaBH₄, was at least 5 fold that of the corresponding untreated “white” porous surface, while the signal-to-noise ratio for the treated tinted substrate is about two times greater than the treated white surface.

### DETAILED DESCRIPTION OF THE INVENTION

### Section I—Definitions

Before describing the present invention in detail, this invention is not necessarily limited to specific compositions, reagents, process steps, or equipment, as such may vary. As used in this specification and the appended claims, the singular forms “a,” “an,” and “the” include plural referents unless the context clearly dictates otherwise. It is also to be understood that the terminology used herein is for the purpose of describing particular embodiments only, and is not intended to be limiting. All technical and scientific terms used herein have the usual meaning conventionally understood by persons skilled in the art to which this invention pertains, unless context defines otherwise.

The term “biological molecule” or “biomolecule” refers to any kind of biological entity, including, such as, oligonucleotides, DNA, RNA, peptide nucleic acid (PNA), peptides, polypeptides, protein domains, proteins, fusion proteins, antibodies, membrane proteins, lipids, lipid membranes, cellular membranes, cell lysates, oligosaccharides, or polysaccharides, or lectins.

The term “biospot” or “microspot” refers to a discrete or defined area, locus, or spot on the surface of a substrate, containing a deposit of biological or chemical material.

The term “complement” or “complementary” refers to the reciprocal or corresponding moiety of a molecule to another. For instance, receptor-ligand pairs, or complementary nucleic acid sequences, in which nucleotides on opposite strands that would normally base pair with each other according to Watson-Crick-base pair (A/T, G/C, C/G, T/A) correspondence.

The term “fluid” or “film of fluid” as used herein refers to a material or medium that can flow such as a gas, a liquid, or a semisolid.

The term “functionalization” as used herein relates to modification of a solid substrate to provide a plurality of functional groups on the substrate surface. The phrase “functionalized surface” as used herein refers to a substrate surface that has been modified to have a plurality of functional groups present thereon.

The terms “nucleoside” and “nucleotide” are intended to include those moieties which contain not only the known purine and pyrimidine bases, but also other heterocyclic bases that have been modified. Such modifications include methylated purines or pyrimidines, acylated purines or pyrimidines, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. As used herein, the term “amino acid” is intended to include not only the L-, D- and nonchiral forms of naturally occurring amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine), but also modified amino acids, amino acid analogs, and other chemical compounds which can be incorporated in conventional oligopeptide synthesis, e.g., 4-nitrophenylalanine, isoglutamic acid, isoglutamine, ε-nicotinoyl-lysine, isonipecotic acid, tetrahydroisoquinoleic acid, α-aminoisobutyric acid, sarcosine, citrulline, cysteic acid, t-butylglycine, t-butylalanine, phenylglycine, cyclohexylalanine, β-alanine, 4-aminobutyric acid, and the like.

The term “probe” refers to either a natural or synthetic molecule, which according to the nomenclature recommended by B. Phimister (Nature Genetics 1999, 21 supplement, pp. 1-60.), is immobilized to a substrate surface. The corresponding microspots are referred to as “probe microspots,” and these microspots are arranged in a spatially addressable manner to form a microarray. When the microarray is exposed to a sample of interest, molecules (“targets”) in the sample selectively and specifically binds to their binding partners (i.e., probes) in the microarrays. The binding of a “target” to the microspots occurs to an extent determined by the concentration of that “target” molecule and its affinity for a particular probe microspot.

The term “receptor” refers to a molecule that has an affinity for a ligand. Receptors may be naturally-occurring or man-made molecules. They may be employed in their unaltered state or as aggregates with other species. Examples of receptors which may be employed according to this invention may include, but are not limited to, antibodies, monoclonal antibodies and antisera reactive with specific antigenic determinants, pharamaceutical or toxin molecules, oligonucleotides, polynucleotides, DNA, RNA, peptide nucleic acid (PNA), peptides, polypeptides, protein domains, proteins, fusion proteins, cofactors, lectins, oligosacharides, polysacharides, viruses, cells, cellular membranes, cell membrane receptors, and organelles. Receptors are sometimes referred to in the art as anti-ligands. A “ligand-receptor pair” is formed when two molecules have combined through molecular recognition to form a complex.

The term “sample” as used herein relates to a material or mixture of materials, typically, although not necessarily, in fluid form, containing one or more components of interest.

The term “substrate,” “microarray substrate” or “substrate surface” refers to a solid or semi-solid material that is porous or semi-porous, which can form a stable support for immobilized probe molecules. The substrate surface can be selected from a variety of materials. For instance, the materials may be biological (e.g., plant cell walls), non-biological, organic (e.g., silanes, polylycine, hydrogels), inorganic (e.g., glass, ceramics, SiO₂, gold or platinum, or gold- or platinum-coated), polymeric (e.g., polyethylene, polystyrene, polyvinyl, polyester, etc.), or a combination of any of these, in the form of a slide, plate, film, particles, beads or spheres. Preferably, the substrate surface is two dimensional and relatively flat, and fully porous for the printing of an array of biospots, but may take on alternative surface configurations. For example, the substrate may be textured with raised or depressed regions. Preferably, the substrate surface will have thereon at least one kind of functional or reactive group, which could be amino, carboxyl, hydroxyl, thiol groups, amine-reactive groups, thiol-reactive groups, Ni-chelating groups, anti-His-antibody groups, or the like.

As used herein, the term “target(s),” “target moieties,” “target analyte,” “biological target,” or “chemical target” refers to a solvated particle, molecule or compound of interest in a sample that is to be detected and identified. Suitable targets include organic and inorganic molecules, biomolecules. In a perferred embodiment, the target may be an environmental pollutant (e.g., such as pesticides, insecticides, toxins, etc); a chemical (e.g., solvents, polymers, organic materials, etc); a therapeutic molecule (e.g., therapeutic and abuse drugs, antibiotics, etc); a biomolecule (e.g., hormones, cytokines, proteins, peptides, protein domains, fusion proteins, nucleotides, oligonucleotides, DNA, RNA, peptide nucleotide acids (PNA), genomic DNA, lipids, lipid membranes, carbohydrates, cellular membrane antigens, receptors or their ligands, etc); whole cells (e.g., pathogenic bacteria, eukaryotic cells, etc); a virus; or spores, etc.

### Section II—Description

Porous substrates, like those described in U.S. Patent Publication Nos. 2003-0003474, or 2002-0142339, or PCT Publication No. WO 00/61282, or an article by M. Glazer et al., Colloidal Silica Films for High-Capacity DNA Probe Assays, Chem. Mater. 2001, 13, 4773-4782, the contents of each are incorporated herein by reference, have been in recent years a subject area of activity and development in the solid-phase biological assay field. A porous substrate involves a substantially flat, porous, inorganic layer applied or adhered to a non-porous support. A beneficial characteristic of porous substrates is their enhanced ability to retain nucleic and/or other probe moieties for high-density arrays. The porous surface provides a greater amount of surface area for immobilizing DNA probe molecules, for instance, which increases the density of nucleic acid binding sites per unit cross-sectional area of the substrate. The increased number of possible binding sites per unit area results in greater retention of immobilized nucleotide probes and the emission of an higher signal level when hybridized with target molecules. A porous inorganic surface that is properly treated with a coating of a binding agent, such as a poly-silane or cationic polymer, can also prevent lateral cross-talk.

Porous ceramic or glass substrates for DNA-binding can consistently yield improved performance relative to both other porous and non-porous flat substrates, and can satisfy other requirements such as chemical and mechanical durability. According to a preferred method, the porous surfaces are fabricated by means of a tape-casting or a screen-printing process using respectively a ceramic or glass containing slip or paste/ink. Adjustments in firing temperature, firing time, and size of the ceramic or glass particles can control the size of the microstructures; hence, the porous layer may have a porosity ranging from about zero or one or two percent up to potentially 99%. Preferable porosity may range from about 55% to about 80% or 90%. Tape-cast porous borosilicate glass (Corning Inc., Code 7761) layers on calcium aluminosilicate glass slides (Corning Inc., Code 1737) tend to retain the greatest absolute quantity of nucleotides after printing and through all washing, blocking, hybridizing, and rinsing steps. Printed DNA bound on porous tape-cast borosilicate are accessible for hybridization, and exhibit higher absolute signals and signal-to-noise ratio than achieved for porous glass slides or sol-gel coated slides.

The performance of an HDA depends on several factors, such as composition and purity of the substrate, surface chemistry applied to the substrate, and quality of biological molecules applied at all stages of manufacture and use. Generally, from a device viewpoint, a microarray is a sensor, and its response can be benchmarked using standard criteria. Three reference points of merit for any sensor are detection threshold, sensitivity, and dynamic range. The detection threshold is the level at which the smallest input to the sensor can be detected in the output response. The sensitivity relates the input signal to the output signal of the sensor in the dynamic range. The dynamic range, in combination with the detection threshold, defines an upper limit for the response of the device. Inputs greater than some threshold value do not change the sensor output.

The performance of different types of microrrays can be compared using these criteria. A superior microarray is one with the lowest detection threshold, highest sensitivity, and widest dynamic range. The benefits of lower detection threshold are immediately apparent. Differential expression can be measured for genes expressed at lower concentrations of biological molecules. The accuracy of measurement in testing for differential expression is affected by sensitivity. Higher sensitivity provides greater accuracy, especially at concentrations near the detection threshold. With the higher sensitivity, uncertainty or error in intensity due to factors associated with excitation laser and photomultiplier detector in the scanner can be reduced. Thus, discrimination between smaller concentrations can be made with greater accuracy in differential gene expression. A wider dynamic range is also an attractive feature, which is achievable if provided with properly calibrated and adjusted scanning equipment that is compatible and capable of accommodating high-sensitivity microarrays.

According to the present invention, porous, inorganic substrates can provide significant advantages over prior inorganic and organic substrates for high-density DNA arrays. Porous inorganic substrates for arrays have superior sensitivity and lower detection threshold when compared to flat, nonporous surfaces. Porous inorganic substrates having certain types of microstructure can produce fluorescent-molecule sensitivities of one or more than two orders of magnitude greater than that of a flat, non-porous slide. Sensitivity is an important property for biological applications where detection of fluorescent molecules is required. A substrate with higher sensitivity is attractive for these applications since smaller changes in concentration and possibly lower overall concentrations can be more easily detected. Enhanced sensitivity and lower detection thresholds provide opportunities to reduce cost for the array manufacturer or user. Less material could be printed during manufacture, or the concentration of probes in hybridization solution could be reduced while still maintaining the same level of performance, if not a higher level than that of a flat slide.

Even with such advantageous, researchers have discovered that porous substrates unfortunately exhibit a higher background or auto-fluorescence at certain wavelengths, particular in the visible light spectrum, between about 400 nm to 700 nm, than coated non-porous flat glass substrates. In an effort to overcome this problem, while preserving the beneficial aspects of porous substrates, we have developed the present invention. Auto-fluorescence or intrinsic background of porous glass materials can be reduced effectively, according to the present invention, by doping the glass composition with certain inorganic components to tint the porous glass layer. This would be more attractive to users of porous substrates who want a low background with high signal intensity. Moreover, the present invention can both enhance sensitivity and improve threshold detection of fluorescence markers.

According to the present invention, a colorant incorporated into the composition of the porous inorganic component or coating layer on a porous substrate can reduce the relative level of reflectance and auto-fluorescence. In certain embodiments, cobalt oxide or nickel (II) oxide components, which are used in Black Light Blue glass for conventional lighting applications, were incorporated into a glass composition to achieve a tint. The dark glass frit appears as a grayish layer after its is applied and fired to bond to the underlying non-porous backing or support. Although not intending to be bound by theory, it is believed that enhanced sensitivity in porous inorganic substrates is based upon light scattering. Uncontrolled light scattering, however, is believed to be a concern. For substrates that have yet to be prepped with the biological molecules or probes, the colorant tint according to the invention consistently reduces background issues due to uncontrolled light scattering in a coated or bare porous glass layer.

We evaluated various compositions of glasses. Starting with an example of a base “white” or un-tinted glass from a family of borosilicate or alumino-borosilicate glasses, we modified the original glass composition, provided in Table 1, according to the present invention.

TABLE 1

| | | | Amount |
|---|---|---|---|
| Oxide | wt % | Batch Components | (grams) |
| SiO₂ | 63.3 | Berkley Sand | 1274.10 |
| Al₂O₃ | 7.9 | Alumina | 157.40 |
| B₂O₃ | 17.8 | Boric Oxide | 360.50 |
| CaO | 2.0 | Calcium Carbonate | 70.41 |
| BaO | 4.1 | Barium Carbonate | 106.40 |
| SrO | 0.9 | Strontium Carbonate | 25.70 |
| Sb₂O₃ | 0.9 | Antimony Trioxide | 17.80 |
| MgO | 0.35 | Magnesia | 7.10 |
| K₂O | 2.7 | Potassium Carbonate | 79.10 |

We discovered that glass frits with different cobalt and/or nickel levels appear to work well to suppress background fluorescence. A combination of these dopants or additional other species may be incorporated that will reduce background without affecting the signal intensity of the desired fluorescence used for the target-probe binding assay. Table 2 summarizes the compositional examples for some glasses according to the present invention.

TABLE 2

| Oxide | Ex. 1 | Ex. 2 | Ex. 3 | Ex. 4 | Ex. 5 | Ex. 6 | Ex. 7 |
|---|---|---|---|---|---|---|---|
| SiO₂ | 63.1 | 61 | 59.9 | 55 | 60.4 | 56.5 | 63.35 |
| Al₂O₃ | 7.5 | 7.3 | 7.1 | 6.6 | 7.2 | 6.7 | 7.5 |
| B₂O₃ | 17.7 | 17.4 | 16.8 | 15.7 | 16.9 | 15.8 | 17.76 |
| K₂O | 2.7 | 2.6 | 2.5 | 2.4 | 2.6 | 2.4 | 2.68 |
| MgO | 0.35 | 0.34 | 0.33 | 0.31 | 0.33 | 0.31 | 0.35 |
| CaO | 1.5 | 1.5 | 1.4 | 1.3 | 1.4 | 1.3 | 1.5 |
| SrO | 0.9 | 0.9 | 0.9 | 0.8 | 0.9 | 0.8 | 0.92 |
| BaO | 3.6 | 3.5 | 3.4 | 3.2 | 3.4 | 3.1 | 3.6 |
| Sb₂O₃ | 0..8 | 0.9 | 0.85 | 0.8 | 0.0 | 0.0 | 0.9 |
| Co₃O₄ | 0.8 | 1.6 | 3.0 | 5.6 | 3.0 | 5.7 | 0.4 |
| NiO | 1.0 | 2.0 | 3.8 | 7.1 | 3.8 | 7.1 | 1 |
| other | 0.05 | 1.0 | | 1.0 | | 0.1 | 0.04 |
| Transition | | | | | | | |
| Metals | | | | | | | |

Table 3 presents possible ranges, according to some embodiments, for the major components of the tinted porous glass.

TABLE 3

| wt % | Lower range | Upper range |
|---|---|---|
| SiO₂ | 53 | 67 |
| Al₂O₃ | 5 | 10 |
| B₂O₃ | 12 | 24 |
| K₂O | 0 | 5 |
| MgO | 0 | 1 |
| CaO | 1 | 3 |
| SrO | 0 | 3 |
| BaO | 3 | 7 |
| Sb₂O₃ | 0 | 2 |
| Co₃O₄ | 0 | 2 |
| NiO | 0 | 4 |
| other Transition | 0 | 4 |
| Metals | | |

For tinting agents, the preferred composition would still have cobalt and nickel oxides, but the other transition metal elements such as Fe, V or Cu, which exhibited little or no detectable increase (as 1×3 in slides) in auto-fluorescence can be included as well, although they are not as strongly absorbent of auto-fluorescence as Co and Ni. We observed additional fluorescence from Cr and Mn. Hence, one may exclude these elements. Also, it is possible to make these glasses without the Sb fining agent, and it might even be desirable to do that under some circumstances.

In the working examples, borosilicate glass was selected as the porous layer since borosilicates are transparent and are readily available, although other glasses having similar physical characteristics may be substituted. The glass transition/sintering temperature of the substrate and porous layer should be similar so as to provide for strong adhesion between the two. Also, in the ideal situation, the surface is positively charged in a neutral aqueous solution, so as to aid in attaching the negatively charged DNA molecules.

### Section III—Example

Table 4 provides a comparison between an example of an initial or original “white” borosilicate composition and a similar example according to the invention containing colorant components.

TABLE 4

| | Ex. Initial | Ex. 7 |
|---|---|---|
| wt % | Base | tinted |
| SiO₂ | 64.16 | 63.35 |
| Al₂O₃ | 7.54 | 7.5 |
| B₂O₃ | 17.84 | 17.76 |
| K₂O | 2.68 | 2.68 |
| MgO | 0.35 | 0.35 |
| CaO | 1.51 | 1.5 |
| SrO | 0.92 | 0.92 |
| BaO | 4.1 | 3.6 |
| Sb₂O₃ | 0.9 | 0.9 |
| Co₃O₄ | | 0.4 |
| NiO | | 1 |
| other Transition | | 0.04 |
| Metals | 100 | 100 |

Crushed borosilicate glass particles are sieved and wet-milled to a reduced particle size (average size in the range of about 0.07-3.5 μm). The particles were ball-milled for 24-72 hours using a one gallon bottle (Nalgene) charged with the crushed borosilicate glass, ZrO₂ milling cylinders and filled with isopropanol to about 85 percent full. After milling, the slurry was stirred and then allowed to stand without disturbance for the particles to settle. Settling can further control the size distribution of the glass particles before a binder is added. The liquid slurry was poured from the Nalgene bottle and the isopropanol was evaporated on a hot plate to recover the glass powder. Care was taken no to disturb the sediment at the bottom of the bottle. The average particle size of the borosilicate powder obtained after settling was in the range of about 0.05-1.5 μm. The borosilicate powder was used in preparation of slip for tape casting.

U.S. Pat. No. 5,089,455, incorporated herein by reference, describes in detail the preparation of zirconia based slips for the tape casting of thin zirconia electrolytes such as for fuel cell applications. Preparation of the borosilicate slip for casting of a porous layer was performed in analogous fashion according to the procedure given in that patent. The recipe was adjusted to account for the difference in density of ZrO₂ and borosilicate, and no settling was performed to narrow the particle size distribution. In brief, 100 g of milled borosilicate powder, 90.9 g ethanol, 21.98 g 1-butanol, 5.0 g propylene glycol, 6.25 g distilled water, 2.5 g Emphos, and 1125 g of one cm ZrO₂ milling balls were weighed into a 500 ml nalgene bottle and vibratory milled for 72 hours. The milled slip was poured from the Nalgene bottle without the milling media into a new 250 mL Nalgene bottle. The final step in the preparation of the slip was to add 5.0 g of a 50 w/o mixture of glacial acetic acid and isopropanol, 8.75 g dibutylphthalate, and 15 g polyvinylbutyral, and five or six 1 cm zirconia milling balls. The bottle was then rolled gently at less than 1 rotation per second to thoroughly mix and remove bubbles for at least 72 hours prior to tape casting.

Tape casting of the slips to form the porous substrates for microarrays is relatively straightforward. Using a non-porous understructure made from a calcium aluminosilicate glass (Corning Inc., Code 1737), a panel of glass scored to give 1 inch by 3 inch microscope slides was cleaned on both major surfaces. Using tape casting doctor metal blades of various dimensions, one may create different layer thicknesses of the tinted porous glass layer. For instance, one example used a 4 mil blade, while another with a 2 mil blade, and the last used a 2 mil blade plus a layer of tape. One may first apply a bonding layer to the non-porous surface. This bonding layer, however, is not required if the underlying substrate and the frit or porous layer have a similar coefficient of thermal expansion (CTE), so that the porous layer should not delaminate from the substrate. The coating should be allowed to dry before proceeding. The frit glass slip is cast on top using another tape casting blade. The coated slides were allowed to dry. The actual thickness of the porous layer is not necessarily limiting, in some embodiments. Several repeated applications with the tape casting blade can build the porous layer up to approximately 2.5 mm in thickness. The dried and fired porous layers may be as thick as about 1 mm. The final porous layer in preferred examples range from about 5 or 6 μm to about 100 or 150 μm. More preferably, the thicknesses may range from about 10 to 75 μm or about 15 to 50 μm. A most desired fired thickness for the porous layer is about 30 μm ±5 μm.

Once the porous coating is dry, the 1737-glass panel can be snapped into individual slides and fired. These tape-cast slides were fired on alumina fiber board using an alumina fiber board cover. The coated slides were fired at a temperature that causes the bonding layer to fuse, and the top frit glass to sinter into a porous layer. The exact temperature (e.g., ˜650-735° C.) and duration (e.g., ˜2-3 hours) of firing may vary according to the glass composition or desired characteristics of the porous layer. In general, porosity of the coatings decreases with increasing firing temperature. After firing, the substrates are allowed to cool to ambient temperature for 4 hours.

The example slides along with a sample each of the original white porous slides and a plain uncoated 1737-glass LCD panel were fired at about 705° C., and were scanned after firing to determine the background. Fired slides are translucent and have a hazy appearance due to light scattering. The tinted porous layers have a grayish color. The porous layer should be strongly bonded to the calcium aluminosilicate glass substrate. Larger pores were ˜5 μm, and the smaller pores have an average size of ˜0.5 μm to ˜1.0 μm.

Light scattering due to the difference in refractive index between the pore and the solid material of the porous layer is greatest when the pore size is similar to the wavelength of the fluorescent markers. Typical chemical markers used in biological assays fluoresce in the visible range, 300-800 nm, which includes the size of pores inherent to the tape cast porous layers. It is believed that light scattering generated by the random index variations in the porous layer creates local higher excitation intensity. Unlike in an ordinary flat, nonporous glass slide, where photon excitation has but only one opportunity to interact with a fluorophore molecule, in a porous-coated substrate excitation is scattered multiple times before exiting the porous layer. This light scattering effect may in part be due to the microstructure features of the porous layer such as layer thickness, particle size, particle shape, pore size, pore shape, porosity, continuity of the glass and pore phases, surface density of binding sites, etc. Adjustments of these parameters may optimize the light scattering effect. Thus, a higher rate of light emission from the fluorescent molecules is possible in the porous layer provided that the two-level fluorescent system is not itself saturated. The light scattering effect and enhanced sensitivity disappear on infiltration of the pores of the coating with an index matching fluid such as glycerol.

It is believed that the superior sensor characteristics of a porous slide of the present invention are due to a higher surface area for binding of biological molecules, improved excitation of fluorophore due to scattering of excitation through the porous surface, and rapid hybridization kinetics. The porous surface can have greater density of binding sites per unit area for DNA attachment than a comparable flat nonporous substrate. Hence, a greater absolute number of printed DNA molecules can be retained through all steps of a DNA analysis process. An increase in the absolute number of retained DNA is important, since it minimizes the loss of DNA during the processing steps. Also, since it is proportionate to the absolute number of DNA molecules, the optical signal from the fluorescent tags on both the printed, known DNA strands and any hybridized, unknown strands is strengthened.

Additionally, it is believed that the effective number of binding sites on the substrate increases with decreasing particle size and increasing thickness of the porous layer. Retention of DNA can be enhanced by the microstructural characteristics of a porous, nucleic-acid-binding surface. As stated before, retention of printed DNA through washing, blocking, hybridizing, and rinsing operations is critical. Excessive loss of the printed DNA leads to a low fluorescent signal-to-noise-ratio and lack of confidence in the analysis. A porous surface effectively increases the number and density of possible DNA binding sites per unit area of the cross-section. Moreover, one should keep in mind that the type of surface chemistry, ink composition, print pins size, and ink volume may effect sensitivity, though not light scattering.

To achieve enhanced sensitivity, two other parameters preferably should be satisfied. First, the distribution of the fluorescent molecules on the internal surfaces of the porous glass structure should overlap localized higher excitation intensity. Second, light emitted by the fluorescent molecules should be able to escape the porous structure to be observed and measured. The distribution of fluorescent molecules as a function of depth in the porous coating may have a dramatic effect on sensitivity. One can alter this distribution by modifying the density of binding sites or the number of molecules to be bound that are present in the ink. No matter what the concentration of biological material in printing inks one can achieve heightened sensitivity in the inventive porous substrates relative to conventional substrates.

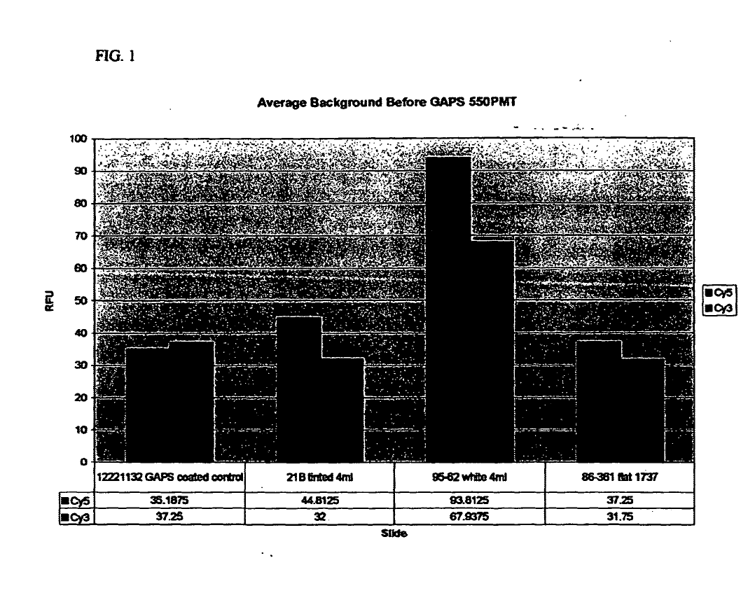

As mentioned, the samples are scanned for background auto-fluorescence after firing. As evident in FIG. 1, the background fluorescence of the tinted porous glass is reduced compared to the original white porous layer by over 50% in terms of relative fluorescence units (RFU). In fact, the auto-fluorescent background on the tinted porous slides is comparable to the two-dimensional flat GAPS coated control slide, and the uncoated 1737-glass LCD panel.

Afterwards, the porous slides were dip coated with a 5% GAPS solution to prepare them for attaching nucleotides. The GAPS coated porous slides were scanned again to compare the auto-fluorescent background due to the coating process. Along with CVD-manufactured control slides, the samples of GAPS-coated, tinted slides were printed with a gene (pBR500mer) DNA. Two inks were used, one containing DMSO/citrate solution and the other a ethylene glycol/water solution, such as described in co-assigned U.S. patent application Ser. No. 10/244898 by S. Pal.

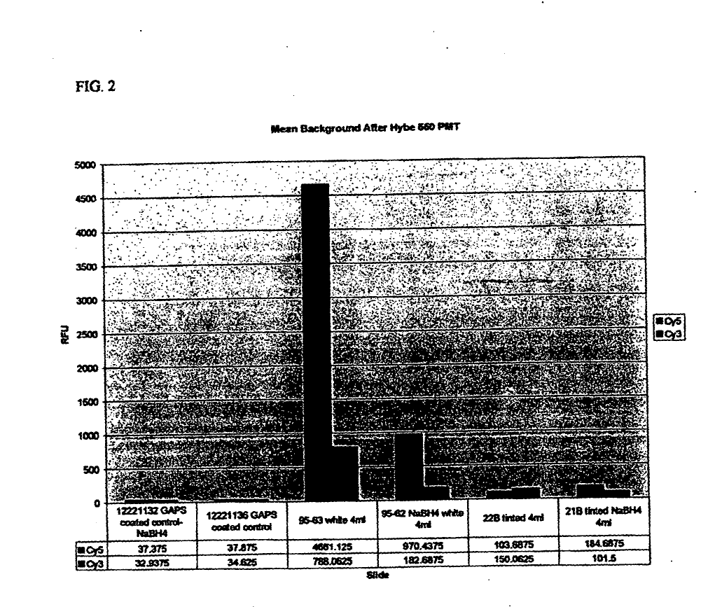

One picomole of Cy3 and Cy5 tagged pBR500 probe was used per slide for hybridization. The hybridization solution used included 25% Formamide, 5×SSC and 0.1%SDS. Salmon Testes DNA was used as the blocker. One of each porous slide was treated with a reducing agent, such as NaBH₄ which is used to decrease Cy3 background signal, and one of each slide were left untreated with the NaBH₄. The microarrays were hybridized over night in a 42° C. water bath. Afterwards, the hybridized slides were washed and scanned using a Genepix 4000B scanner for the analysis. A mean background was calculated. The background reflectance in the tinted porous had been reduced significantly compared to the original white porous glass. The results are represented in FIG. 2, which indicate that the signal to noise ratio is lower in the tinted porous substrate with respect to the Cy5 signal. The tinted porous substrate exhibited less than about 20% or 25% background fluorescence relative to that shown for the “white” porous substrate. In other words, the tinted substrate displayed only about ¼ to about ⅕ of the background of the white substrate. This phenomenon may be attributable to the advantage of a tinted porous glass substrate layer as well as in part, with respect to the Cy3 signal, to the NaBH₄ treatment reducing organic auto-fluorescence impurities due to the GAPS coating.

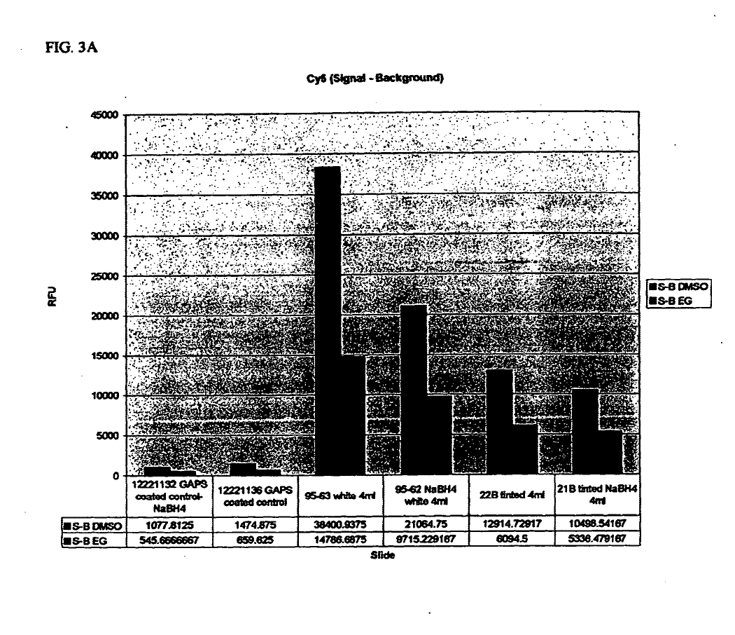

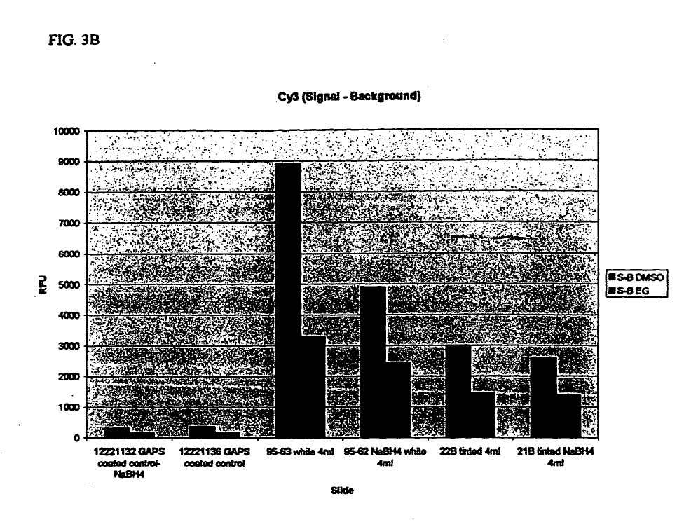

As FIGS. 3A and 3B show, when compared to the original white porous glass, the net signal (Mean signal—Mean background) for both Cy3 and Cy5 fluorescence is lower in the tinted porous glass.

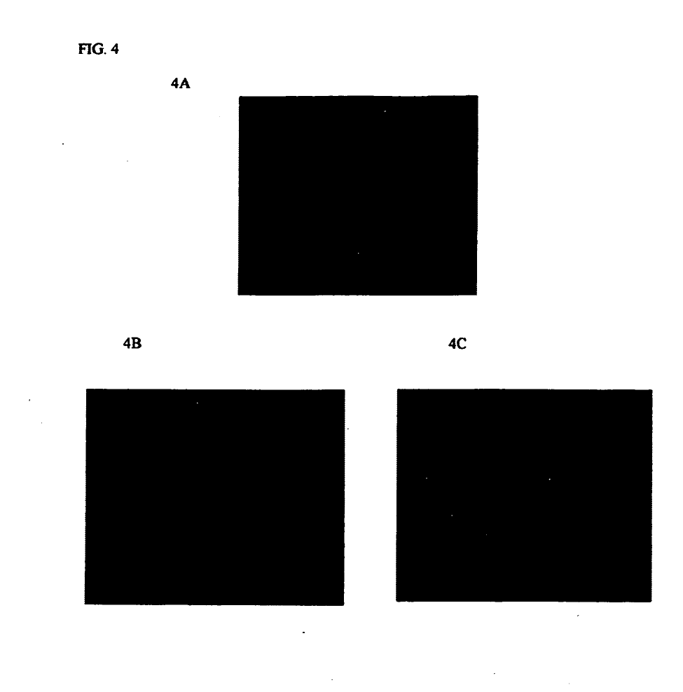

Arranged together for convenience of comparison, FIGS. 4A-4C are images of three substrates after firing. FIG. 4A presents the surface of a typical 1737-glass panel, which serves here as a control. Notice that the non-porous substrate exhibits relatively minor levels of auto-fluorescence as data in the foregoing graphs support. An example of the tinted porous glass of the present invention in FIG. 4B stands rather favorably in comparison, to the non-porous flat glass surface in terms of relative background signal. By contrast, the “white” porous glass substrate of FIG. 4C exhibits a relatively high degree of background fluorescence, as data in the aforementioned graphs confirm. The “white” porous surface shows not only a few luminous spots, but also some detectable speckles over its surface, especially along the left side of the image.

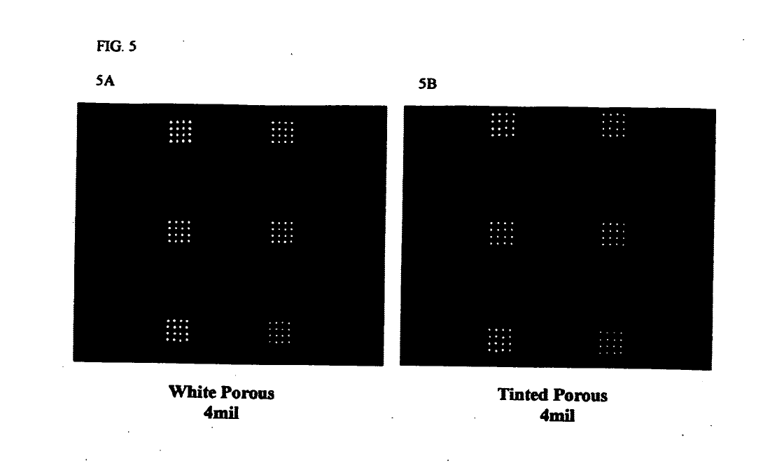

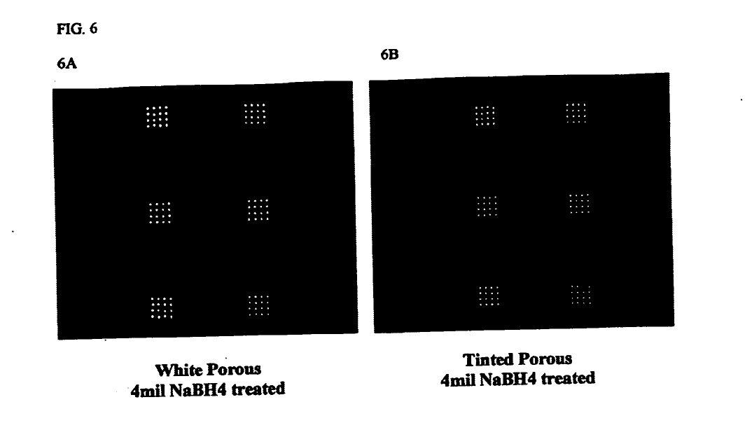

Comparing side-by-side false color images of samples of “white” and tinted porous substrates under both Cy3 and Cy5 confirms the fact that tinted substrates are inherently superior—not merely manipulation of parameters—and an improvement in reducing background. FIGS. 5A, 5B, 6A, and 6B are images from a GenePix scanner. As one can see in FIG. 5A, the “white” porous substrate, not treated with a reducing agent, NaBH₄, exhibits a noticeable and significant amount of background fluorescence relative to tinted, porous glass substrate of FIG. 5B. A hazy, whitish background is evident and surrounds the microarray dot areas of the uncolored, porous glass substrate, whereas for the tinted species, the background fluorescence is barely noticeable. This background phenomenon and contrast is still apparent when comparing FIGS. 6A versus 6B, even after treatment with, NaBH₄, to remove residual organic contaminants on the substrate's surface. The uncolored porous substrate retains some degree of fluorescence that is higher than the auto-fluorescence level for the tinted, porous glass substrate. The comparison of images shows that a tinted porous substrate reduces background regardless of whether or not the slide was treated with NaBH₄.

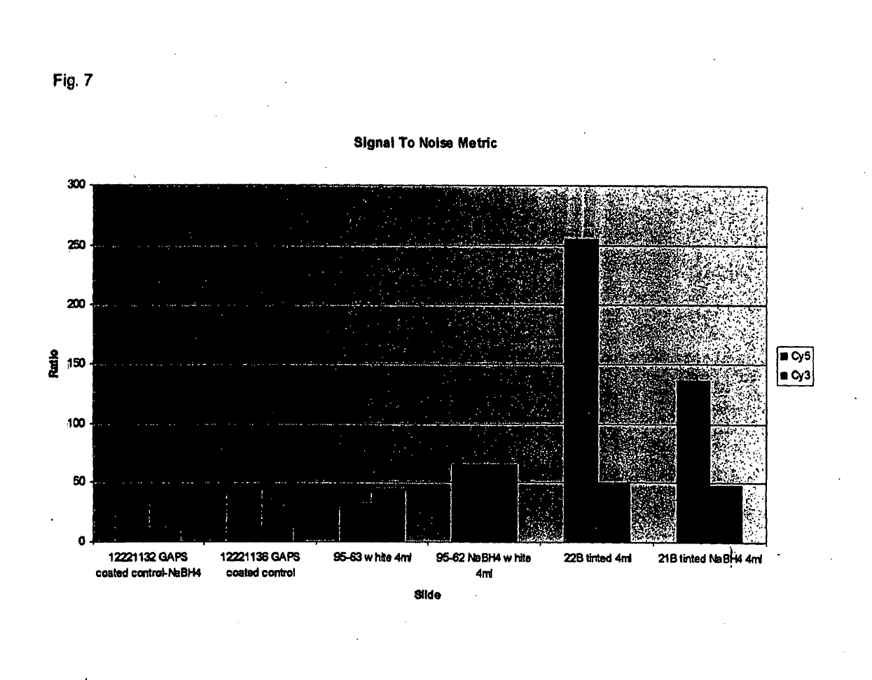

The colorant dopants in the tinted porous glass cause a substantial reduction in the Cy5 background. This reduction in background signal significantly impacts the relative signal-to-noise ratio. That signal to noise ratio is calculated as net signal divided by the background signal. Some results of the metric calculation are shown in FIG. 7, in which the data suggest that signal-to-noise ratio for tinted porous substrates improves overall performance by reducing the high background fluorescence observed in the white porous glass.

### Section IV—Examples of other Array Species & their Associated Binding-Assays

The devices of the present invention help the performance of binding assays detect targets in samples. The target analytes preferably bind with probe molecule(s) immobilized on a surface of the substrate. Even though the inventive device and method has been described in the context of nucleotide reactions for illustrative purposes, the present invention is not necessarily limited only to nucleotide hybridization assays. Alternate applications that may benefit from the present invention may include other biological binding-assay formats. The kinds of probe molecules which may be immobilized on a porous layer may be selected from a variety of biological or chemical species or molecules. For instance, the probe molecules may include proteins, peptides, polypeptides, protein-membranes, which may be useful for the emerging field of proteomics, G-coupled protein receptors, gangliosides, cells or cell membranes, cell-lysate, or protein-small molecule ligands, for drug compound molecule interactions. A description of several examples of different kinds of microarrays and their various uses follow.

When a set of probe nucleic acid molecules with known sequences are tethered or immobilized onto a surface in confined locations in a DNA microarray, the target analyte can be a nucleic acid. The target sequence may be a portion of a gene, a regulatory sequence, genomic DNA, cDNA, RNA including mRNA and rRNA, or others. The target sequence is preferred used to be in a single-stranded format; however, the target sequence in a double stranded conformation (e.g., genomic DNA) may be used after denaturation. The target sequence is preferably labeled with a detectable moiety or moieties, such as fluorescence dye molecule(s) to allow detection of the binding of the target sequence to the probe microspots directly using fluorescence imaging techniques, or with biotin moieties in which a sequential detection step using labeled anti-biotin or anti-biotin coated gold nanoparticle is required for detection the binding of the target sequence to the probe microspots (Bao et al. Anal. Chem. 2002, 74, 1792-1797). A “probe nucleic acid” or “probe sequence” refers to a nucleic acid sequence with known sequence or defined sequence. Preferably, the probe nucleic acid is a cDNA, a oligonucleotide with defined sequence, or a modified oligonucleotide with defined sequence. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 1993, 49,1925), peptide nucleic acid backbones and linkages (Egholm, J. Am. Chem. Soc. 1992, 114,1895); Nielsen, Nature, 1993,365,566). As will be appreciated by those in the art, all of these nucleic acid analogs may find use in the present invention.

According to an embodiment, a pharmacological compound or ligand may be the target compound or ligand when using a probe protein microarray. A “target compound” or “target ligand” in this context refers to a chemical or biochemical or biological compound whose identity, abundance, or binding affinity and specificity is to be detected. The target compound can be synthetic, naturally occurring, or biological produced. The target compound may be a street drug of abuse, a pharmaceutical drug candidate, a chemical (an organic or inorganic compound, including ionic salt), a biochemical (e.g., synthetic lipids, oligosaccharides, peptides, amino acids, nucleotides, nucleosides, etc), or a biological (e.g., a naturally occurring lipids, a protein, an antigen, an antibody, a growth factor, etc.). The target compound may be an activator, an inhibitor, an effector, a binding partner, or an enzyme substrate of the probe protein(s). The target compound can be part of a selected or random compound library. A “probe protein” or “probe polypeptide” refers to a polypeptide with a known sequence. The probe proteins may be obtained from natural sources or, optionally, be overexpressed using recombinant DNA methods. The probe proteins may be either purified using conventional approaches or un-purified (e.g., cell lysates). The probe protein includes, but not limited to, intracellular proteins, cell surface proteins, soluable proteins, toxin proteins, synthetic peptides, bioactive peptides, and protein domains. Examples of intracellular proteins include, but are not limited to: oxidoreductases, transferases, hydrolases, lyases, isomerases, ligases, kinases, phosphoproteines, and mutator transposons, DNA or RNA associated proteins (for example, homeobox, HMG, PAX, histones, DNA repair, p53, RecA, robosomal proteins, etc.), electron transport proteins (e.g., flavodoxins); adaptor proteins; initiator caspases, effector caspases, inflammatory caspases, cyclins, cyclin-dependent kinases, cytokeletal proteins, G-protein regulators, small G proteins, mitochondria-associated proteins, PDZ adaptor proteins, PI-4-kinases, etc. Recombinant proteins of unknown functions may also be used. Applicable cell surface proteins include, but are not limited to: G-protein coupled receptors (e.g., aderenergic receptor, angiotensin receptor, cholecystokinin receptor, muscarinic acetylcholine receptor, neurotensin receptor, galanin receptor, dopamine receptor, opioid receptor, erotonin receptor, somatostatin receptor, etc), G proteins, ion-channels (e.g., nicotinic acetylcholine receptor, sodium and potassium channels, etc), receptor tyrosine kinases (e.g., epidermal growth factor (EGF) receptor), immune receptors, integrins, and other membrane-bound proteins. Mutants or modifications of such proteins or protein functional domains or any recombinant forms of such proteins may also be used. Toxin proteins include, but are not limited to, cholera toxin, tetanus toxin, shiga toxin, heat-labile toxin, botulinum toxin A & E, delta toxin, pertussis toxin, etc. Toxin domains or subunits may also be used. In this embodiment, the probe protein microarrays may be used to identify small molecule-binding proteins (Zhu, H., Bilgin, M., Bangham, R., Hall, D., Casamayor, A., Bertone, P., Lan, N., Jansen, R., Bidlingmaier, S., Houfek, T., et al. “Global analysis of protein activities using proteome chips” Science 2001, 293, 1201-2105), or used to measure protein kinase activities (Houseman, B. T., Huh, J. H., Kron, S. J., Mrksich, M. “Peptide chips for the quantitative evaluation of protein kinase activity”, Nature Biotechnology 2002, 20, 270-274), or used to profile compounds for pharmacological uses (binding affinity, selectivity, and specificity) and screen compounds (Fang, Y., et al. “Membrane protein microarrys”. J.Am. Chem.Soc. 2002, 124, 2394-2395; and Fang, Y. et al. “Membrane biochips” BioTechniques, 2002, 33, S62-S65).

In a further embodiment, the target analyte may be an antigen, a hormone, a cytokine, an immune antibody, a protein, a lipid, or a mixture of un-purified cell lysate, when a probe antibody microarray is used. By “target biologicals” herein means a biological from a biofluid or an organelle or a living cell whose identity/abundance is be detected. The probe antibody includes, but not limited to, an immunoglobulins (e.g, IgEs, IgGs and IgMs), a therapeutically or diagnostically relevant antibodies (e.g., antibodies to human albumin, apolipoproteins including apolipoprotein E, human chorionic gonadotropin, cortisol, a-fetoprotein, thyroxin, thyroid stimulating hormone, antithrombin; antibodeis to antieptileptic drugs (phenytoin, primidone, carbariezepin, ethosuximide, valproic acid, and phenobarbitol), cardioactive drugs (digoxin, lidocaine, procainamide, and disopyramide), bronchodilators (theophylline), antibiotics (chloramphenicol, sulfonamides), antidepressants, immunosuppresants, abused drugs (amphetamine, methamphetamine, cannabinoids, cocaine and opiates)), a antibody to any viruses (e.g., antibodies to orthomyxovi ruses such as influenza virus, paramyxoviruses (e.g., respiratory syncytial virus, mumps virus, measles virus), adenoviruses, rhinoviruses, coronaviruses, reoviruses, togaviruses (e.g., rubella virus), parvoviruses, poxviruses (e.g., variola virus, vaccinia virus), enteroviruses (e.g., poliovirus, coxsackievirus), hepatitis viruses (including A, 6 and C), herpesviruses (e.g., Herpes simplex virus, varicella-zoster virus, cytomegalovirus, Epstein-Barr virus), rotaviruses, Norwalk viruses, hantavirus, arenavirus, rhabdovirus (e.g., rabies virus), retroviruses (including HIV, HTLV-I and -11), papovaviruses (e.g., papillomavirus), polyornaviruses, and picornaviruses, and the like), and anthrax, etc.), an antibody to bacteria (e.g., antibodies to a wide variety of pathogenic and non-pathogenic prokaryotes of interest including Bacillus; Vibrio, e.g., V. cholerae; Escherichia, e.g., Enterotoxigenic E. coli, Shigella, e.g., S. dysenteriae; Salmonella, e.g., S. typhi; Mycobacterium e.g., M. tuberculosis, M. leprae; Clostridium, e.g., C. botulinum, C. tetani, C. difficile, C.perfringens; Cornyebacterium, e.g., C. diphtheriae; Streptococcus, S. pyogenes, S. pneumoniae; Staphylococcus, e.g., S. aureus; Haemophilus, e.g., H. influenzae; Neisseria, e.g., N. meningitidis, N. gonorrhoeae; Yersinia, e.g., Y. lamblia, Y. pestis; Pseudomonas, e.g., P. aeruginosa, P. putida; Chlamydia, e.g., C. trachomatis; Bordetella, e.g., B. pertussis; Treponema, e.g., T. palladium; and the like)), an antibody to bacteria toxin (e.g., antibodies to diphtheria toxin, anthrax toxin, tetrodotoxin, saxitoxin, bactrachotoxin, grayanotoxin, veratridine, actonitine, scorpion, sea anemone venom, scorpion charybdotxins, dendrotoxins, hanatoxins, sea anemone toxins, hololena, calcicludine, bungarotoxin, cholera toxin, conantokin, etc).

In certain embodiments, the probe antibody arrays may be used for protein profiling, measurement of protein abundance in blood, measurement of cytokine abundances, detection of bacteria toxins in samples (such as environmental water, or food resources), as well as capture of leukocytes/phenotyping leukemias. These target species may be present in any number of different sample types, including, but not limited to, bodily fluids including blood, lymph, saliva, vaginal and anal secretions, urine, feces, perspiration and tears, and solid tissues, including liver, spleen, bone marrow, lung, muscle, brain, etc. Conversely, the “probes” can also be antigens, in which the antigen arrays may be used for reverse immunoassay to measure immuno-antibodies and allergens.

In another embodiment, a carbohydrate microarray having oligosaccharides or polysaccharides immobilized on to a surface at defined locations may be used to detect carbohydrate-binding protein target(s) in a sample (Fukui, S., Feizi, T., Galustian, C., Lawson, A. M., and Chai, W. “Oligosaccharide microarrays for high-throughput detection and specificity assignments of carbohydrate-protein interactions” Nature Biotechnology, 2002, 20, 1011-1017), or for identifying cross-reactive molecular markers of microbes and host cells (Wang, D., Liu, S., Trummer, B. J., Deng, C., and Wang, A., “Carbohydrate microarrays for recognition of cross-reactive molecular markers of microbes and host cells” Nature Biotechnology, 2002, 20, 275-281), or for identifying specific viruses or bacteria or spores.

The present invention has been described both in general and in detail by way of examples. Persons skilled in the art will understand that the invention is not limited necessarily to the specific embodiments disclosed. Modifications and variations may be made without departing from the scope of the invention as defined by the following claims or their equivalents, including equivalent components presently known, or to be developed, which may be used within the scope of the present invention. Hence, unless changes otherwise depart from the scope of the invention, the changes should be construed as being included herein.

1. A porous substrate comprising: a support; and a porous region on a surface of said support, the porous region being of primarily inorganic material and having a surface upon which a number of probe molecules can be immobilized, the porous region having a tint and exhibits a reduced level of auto-fluorescence of at least about 15% relative to a comparable non-tinted porous substrate surface.

2. The porous substrate according to claim 1, wherein said porous region having a tint that reduces relative auto-fluorescence levels by at least about 20-25% over said non-tinted porous substrate surface.

3. The porous substrate according to claim 2, wherein said porous region having a tint that reduces relative auto-fluorescence levels by at least about 50% over said non-tinted porous substrate surface.

4. The porous substrate according to claim 1, wherein said porous region exhibits a reduced relative auto-fluorescence level in RFU of at least an order of magnitude over said non-tinted porous substrate surface.

5. The porous substrate according to claim 1, wherein said reduction in auto-fluorescence is over a wavelength range from about 400 nm to about 720 nm.

6. The porous substrate according to claim 5, wherein said reduction in auto-fluorescence is over a wavelength range from about 420 nm to about 700 nm.

7. The porous substrate according to claim 1, wherein said tinted porous region has a colorant component including a transition metal ion.

8. The porous substrate according to claim 1, wherein said tinted porous region has a colorant component incorporated in a composition in weight percent consisting essentially of:

| Oxide | wt. % |
|---|---|
| SiO₂ | 53-67 |
| Al₂O₃ | 3-10 |
| B₂O₃ | 12-24 |
| K₂O | 0-5 |
| MgO | 0-2 |
| CaO | 0.5-3 |
| SrO | 0-3 |
| BaO | 2-7 |
| Sb₂O₃ | 0-2 |

and at least one of the following either individually or in combination

| | |
|---|---|
| Co₃O₄ | 0.1-9 |
| NiO | 0.1-10 |
| RₓO_{y} | 0-10 |

wherein R is a transition metal, and x and y are each ≧0.

9. The porous substrate according to claim 8, wherein said R is selected from the group consisting of Fe, V, and Cu.

10. The porous substrate according to claim 1, wherein said porous region has a composition consisting essentially of:

| Oxide | wt. % |
|---|---|
| SiO₂ | 55-65 |
| Al₂O₃ | 4-9 |
| B₂O₃ | 14-21 |
| K₂O | 1-5 |
| MgO | 0.1-2 |
| CaO | 1-2.5 |
| SrO | 0.5-1.75 |
| BaO | 3-5 |
| Sb₂O₃ | 0-2 |

and at least one of the following, either individually or in combination,

| | |
|---|---|
| Co₃O₄ | 0.1-8 |
| NiO | 0.1-10 |
| RₓO_{y} | 0-10 |

wherein R is a transition metal selected from the group consisting of Fe, V, and Cu, and x and y are each ≧0.

11. The porous substrate according to claim 8, wherein said glass composition is chemically and mechanically durable, and has a coefficient of thermal expansion (CTE) of between about 35-44×10⁻⁷/° C.

12. The porous substrate according to claim 11, wherein said glass composition has a CTE of about 38-40×10⁻⁷/° C.

13. The porous substrate according to claim 1, wherein before a GAPS-coating process, said tinted region has an average auto-fluorescence background for Cy3 and Cy5 channels of up to about 50% RFU of said un-tinted porous substrate.

14. The porous substrate according to claim 1, wherein a number of biological or chemical probes are attached at defined locations on or within said tinted porous layer.

15. The porous substrate according to claim 13, wherein said defined locations of probes assume a microarray format of at least one microspot per cm².

16. The porous substrate according to claim 13, wherein said defined locations of probes assume a microarray format of at least 10 microspots per cm².

17. The porous substrate according to claim 1, wherein said probe molecules include at least one kind of species selected from the following: oligonucleotides, nucleotides, nucleosides, DNA, RNA, peptide nucleic acid (PNA), peptides, polypeptides, protein domains, proteins, fusion proteins, antibodies, protein-membranes, G-coupled protein receptors, gangliosides, lipids, lipid membranes, cells or cell membranes, cell-lysate, or protein-small molecule ligands.

18. A tool for performing biological or chemical assays, the tool comprises a non-porous support; and a porous region on a surface of said support, the porous region being of primarily inorganic material and having a surface upon which a number of probe molecules may be immobilized, the porous region having a tint and exhibits a reduced level of auto-fluorescence of at least about 15% relative to a comparable non-tinted porous substrate surface.

19. The tool according to claim 18, wherein said porous region having a tint that reduces relative auto-fluorescence levels by at least about 20-25% over said non-tinted porous substrate surface.

20. The tool according to claim 18, wherein said tinted porous region has a colorant component including a transition metal ion.

21. The tool according to claim 18, wherein said tinted porous region has a colorant component incorporated in a composition in weight percent consisting essentially of:

| Oxide | wt. % |
|---|---|
| SiO₂ | 53-67 |
| Al₂O₃ | 3-10 |
| B₂O₃ | 12-24 |
| K₂O | 0-5 |
| MgO | 0-2 |
| CaO | 0.5-3 |
| SrO | 0-3 |
| BaO | 2-7 |
| Sb₂O₃ | 0-2 |

and at least one of the following either individually or in combination

| | |
|---|---|
| Co₃O₄ | 0.1-9 |
| NiO | 0.1-10 |
| RₓO_{y} | 0-10 |

wherein R is a transition metal, and x and y are each ≧0.

22. The tool according to claim 21, wherein said R is selected from the group consisting of Fe, V, and Cu.

23. The tool according to claim 18, wherein said probe molecules are biological or chemical molecules, including at least one kind of the following: oligonucleotides, nucleotides, nucleosides, DNA, RNA, peptide nucleic acid (PNA), peptides, polypeptides, protein domains, proteins, fusion proteins, antibodies, gangliosides, membrane proteins, lipids, lipid membranes, cellular membranes, cell lysates, oligosaccharides, or polysaccharides, or lectins.
